(19) United States
(12) Patent Application Publication
(10) Pub. No.: US 2018/0256905 A1
(43) Pub. Date: Sep. 13, 2018

(54) Multi-Site Ultrasonic Wireless Pacemaker-Defibrillator

(71) Applicants: Sapienza University of Rome, Rome (IT); Northeastern University, Boston, MA (US)

(72) Inventors: Pietro FRANCIA, Rome (IT); Tommaso MELODIA, Newton, MA (US); Matteo RINALDI, Boston, MA (US)

(21) Appl. No.: 15/910,657

(22) Filed: Mar. 2, 2018

Related U.S. Application Data
(60) Provisional application No. 62/466,176, filed on Mar. 2, 2017.

(51) Int. Cl. A61N 1/372; A61N 1/365; A61N 1/362; A61N 1/39; A61N 1/378

(52) U.S. Cl. CPC A61N 1/37217; A61N 1/36585; A61N 1/3655; A61N 1/36564; A61N 1/3621; A61N 1/3622; A61N 1/3956; A61N 1/3787; A61N 1/3624; A61N 1/36507; A61N 1/39622

(57) ABSTRACT

A system and method to monitor and control heart rhythms using ultrasonic signals, including providing pacing and defibrillation therapy, are provided. A device for monitoring and controlling heart rhythms includes an intra-cardiac implantable device having an ultrasonic transducer to receive and/or transmit ultrasonic signals, and pacing circuitry to convert an acoustic signal into an electrical signal to stimulate or control a cardiac rhythm

### CROSS REFERENCE TO RELATED APPLICATIONS

This application claims priority under 35 U.S.C. § 120 of U.S. Provisional Application No. 62/466,176, filed on Mar. 2, 2017, entitled “Multi-Site Ultrasonic Wireless Pacemaker-Defibrillator,” the disclosure of which is hereby incorporated by reference.

### STATEMENT REGARDING FEDERALLY SPONSORED RESEARCH OR DEVELOPMENT

The invention was developed with financial support from Grant Nos. CNS-1458019 and CNS-1618731 from the National Science Foundation. The U.S. Government has certain rights in the invention.

### BACKGROUND

Sudden cardiac death (SCD) accounts for hundreds of thousands of deaths each year in the United States. The underlying mechanism is sudden onset of lethal cardiac arrhythmias (i.e., ventricular tachycardia or ventricular fibrillation). The trans-venous implantable cardioverter-defibrillator (TV-ICD) is a lifesaving device providing automatic arrhythmia detection and early high-energy defibrillation or fast pacing (anti-tachycardia pacing) that has proven its safety and effectiveness in the last three decades in over one million patients. Additionally, the TV-ICD delivers conventional wired lead-based pacing (pacemaker function) to treat symptomatic bradycardia. Last, the TV-ICD is used in current practice to provide synchronous pacing of the right and left ventricles of the heart (cardiac resynchronization therapy, CRT) through multiple implantable leads in patients with heart failure and cardiac dyssynchrony, a pathological condition characterized by non-synchronous contraction of cardiac walls.

However, implanting trans-venous leads, as required in current TV-ICD practice, comes with significant risks, including pneumothorax, cardiac tamponade, upper extremity deep vein thrombosis, and pulmonary embolus. Moreover, as survival improves in ICD population, the long-term risks of lead malfunction and bloodstream infections become of greater concern. Paradoxically, patients gathering the highest survival benefit from the ICD are most exposed to long-term complications. The leads are often the most vulnerable components of the device, as they can become insulated from the system, fracture, or cause infections. Typically an intravascular polyurethane- or silicone-coated conductor, the implanted lead is subject to motion close to the tricuspid valve with each cardiac systole and is therefore subject to constant mechanical stress. The lead failure rates are close to 40% at 5 years.

Recent advances in battery and electronics miniaturization have made it possible to develop leadless pacemakers that can be completely implanted inside the right ventricle. Two entirely implantable pacemaker systems recently became available: the Nanostim™ Leadless Pacemaker System (St. Jude Medical, Sylmar, Calif., USA) and the Micra™ Transcatheter Pacing System (Medtronic, Minneapolis, Minn., USA). Of note, the absence of a surgically created generator pocket and lack of trans-venous leads connecting this pocket to the heart eliminate the main sources of complications associated with conventional pacemaker implantation. A device for leadless left ventricular pacing has been introduced for patients with an indication to CRT. The WiCS™ (Wireless Cardiac Stimulation, EBR Systems) system performs left ventricular endocardial pacing by transmitting acoustically energy from a subcutaneous transmitter unit to an endocardial receiver unit. WiCS detects right ventricular pacing provided by a co-implanted pacemaker, CRT or ICD and delivers a synchronized left ventricular stimulus. Current leadless pacemakers suffer from some limitations; they are able to perform pacing without the need for wires based on only one individual sensing site, and they are unable to deliver defibrillation therapy. Both the Nanostim™ and Micra™ pacemakers are intended only for patients with an indication for a single-chamber pacemaker. Synchronous atrio-ventricular pacing for the treatment of bradyarrhythmia requiring dual-chamber pacing is unavailable.

As far as prevention of sudden cardiac death is concerned, a new ICD providing high-energy defibrillation therapy via an entirely subcutaneous array (the subcutaneous ICD, S-ICD®, Boston Scientific) has been introduced. The S-ICD is equipped with an extracardiac, extrathoracic subcutaneous electrode. The 8-cm defibrillation coil lies directly between two sensing electrodes and the S-ICD generator acts as the third electrode used for sensing and defibrillation. By eliminating the need for lead placement in the heart, the S-ICD is expected to significantly reduce these complications. However, because of the lack of intra-cardiac electrodes, the S-ICD is unable to pace the heart. Thus, it cannot deliver standard anti-bradycardia pacing, anti-tachycardia pacing and cardiac resynchronization therapy.

### SUMMARY

A wireless ultrasonically networked pacemaker/defibrillator system is provided based on multiple leadless intra-cardiac sensors and actuators. The system can be completely leadless, yet can provide functionalities offered by standard implantable defibrillators and pacemakers through ultrasonic wireless data links. In some embodiments, the system can provide capabilities including, for example and without limitation, monitoring of cardiac contractility and kinesis; detecting the origin of ventricular tachycardia or fibrillation; providing leadless anti-tachycardia pacing for rapid-rate life-threatening ventricular arrhythmia; providing leadless anti-bradycardia pacing; deliver leadless multi-site cardiac resynchronization therapy; and providing defibrillation therapy.

Embodiments of a system for monitoring and controlling heart rhythms can include a network of implantable devices comprising at least a first intra-cardiac implantable device implantable in an atrium or a ventricle of a heart comprising an ultrasonic transducer operative to receive ultrasonic signals, and pacing circuitry operative to convert an acoustic signal into an electrical signal to stimulate or control a cardiac rhythm; and a second implantable device comprising an ultrasonic transducer operative to transmit ultrasonic signals to the first intra-cardiac implantable device to stimulate or control the cardiac rhythm.

Embodiments of a method of monitoring and controlling heart rhythms are provided, including implanting the network of implantable devices in a subject in need thereof; and sensing or controlling a heart rhythm by at least the first intra-cardiac implantable devices.

Embodiment of a system for monitoring and controlling heart rhythms are provided, including a network of implantable devices comprising at least a right atrial sensing and pacing device implantable in a right atrium of a heart comprising an ultrasonic transducer operative to transmit ultrasonic signals; and a right ventricular intra-cardiac sensing and pacing device implantable in a right ventricle of the heart, comprising an ultrasonic transducer operative to receive ultrasonic signals from the right atrial sensing and pacing device to stimulate or control the cardiac rhythm, and pacing circuitry operative to convert an acoustic signal into an electrical signal to stimulate or control a cardiac rhythm.

Embodiments of a system for monitoring and controlling heart rhythms are provided, including a network of implantable devices comprising at least a plurality of intra-cardiac left ventricular pacing devices implantable in a left ventricle of the heart, each left ventricular pacing device comprising an ultrasonic transducer operative to receive ultrasonic signals, and pacing circuitry operative to convert an acoustic signal into an electrical signal to stimulate or control a cardiac rhythm; and a right ventricular sensing and pacing device implantable in a right ventricle of the heart comprising an ultrasonic transducer operative to transmit ultrasonic signals to the plurality of intra-cardiac left ventricular pacing devices to stimulate or control the cardiac rhythm.

Embodiments of a system for monitoring and controlling heart rhythms are provided, including a network of implantable devices comprising at least a plurality of intra-cardiac implantable devices each implantable in an atrium or a ventricle of a heart and comprising an ultrasonic transducer operative to receive ultrasonic signals, and pacing circuitry operative to convert an acoustic signal into an electrical signal to stimulate or control a cardiac rhythm; and a subcutaneously implantable central unit comprising a processing unit, the processing unit including one or more processors and memory, an ultrasonic transducer to transmit and receive ultrasonic signals to the plurality of intra-cardiac implantable devices.

Embodiments of a device for monitoring and controlling heart rhythms are provided, including an intra-cardiac implantable device implantable in an atrium or a ventricle of a heart comprising an ultrasonic transducer operative to receive ultrasonic signals, and pacing circuitry operative to convert an acoustic signal into an electrical signal to stimulate or control a cardiac rhythm.

Embodiments of a micro-electromechanical piezoelectric ultrasonic transducer device are providing, including a piezoelectric layer having first and second opposed surfaces, the piezoelectric layer supported along a fixed boundary by a substrate, the piezoelectric layer deflectable out of a plane; a first electrode disposed on the first surface of the piezoelectric layer, and a second electrode disposed on the second surface of the piezoelectric layer. The piezoelectric layer is operative to deflect out of the plane by an incoming pressure wave and to deflect out of plane by a voltage applied across the first and second electrodes. Circuitry in electrical communication with the first and second electrodes can convert a deflection of the piezoelectric layer into an electric signal or to apply a voltage across the first and second electrodes to force a deflection of the piezoelectric layer.

Other aspects include the following:

1. A system for monitoring and controlling heart rhythms, comprising:

a network of implantable devices comprising at least:
a first intra-cardiac implantable device implantable in an atrium or a ventricle of a heart comprising an ultrasonic transducer operative to receive ultrasonic signals, and pacing circuitry operative to convert an acoustic signal into an electrical signal to stimulate or control a cardiac rhythm; and
a second implantable device comprising an ultrasonic transducer operative to transmit ultrasonic signals to the first intra-cardiac implantable device to stimulate or control the cardiac rhythm.
   2. The system of embodiment 1, wherein the first intra-cardiac implantable device comprises a right ventricular intra-cardiac implantable sensing and pacing device implantable in a right ventricle of the heart; and

the second implantable device comprises a right atrial sensing and pacing device implantable in a right atrium of the heart.

3. The system of embodiment 2, wherein the right atrial sensing and pacing device and the right ventricular sensing and pacing device are operable to communicate with each other via ultrasonic signals.
4. The system of any of embodiments 2-3, wherein the right atrial sensing and pacing device is operative to sense spontaneous atrial electrical activity in the heart if an intrinsic heart rate is above a predetermined pacing lower rate, or pace the right atrium if the intrinsic heart rate is below the predetermined pacing lower rate.
5. The system of any of embodiments 2-4, wherein the right atrial sensing and pacing device is further operative to transmit an ultrasonic signal to the right ventricular sensing and pacing device to trigger a determined atrio-ventricular delay interval, and the right ventricular sensing and pacing device is operative to inhibit pacing if the spontaneous ventricular electrical activity of the heart occurs within the delay interval and to deliver pacing if the spontaneous ventricular activity does not occur within the delay interval.
6. The system of embodiment 5, wherein the delay interval ranges from about 100 ms to about 400 ms.
7. The system of any of embodiments 1-6, wherein the first intra-cardiac implantable device comprises a plurality of intra-cardiac left ventricular pacing devices implantable in a left ventricle of the heart; and

the second implantable device comprises a right ventricular sensing and pacing device implantable in a right ventricle of the heart.

8. The system of embodiment 7, wherein the right ventricular sensing and pacing device is operative to transmit instructions via ultrasonic signals to pace each of the plurality of left ventricular pacing devices.
9. The system of embodiment 8, wherein the right ventricular sensing and pacing device is operative to transmit instructions to focus pacing the left ventricular pacing device closest to a determined origin of arrhythmia of the heart.
10. The system of any of embodiments 7-9, wherein each of the left ventricular pacing devices is powered by ultrasonic signals transmitted from the right ventricular sensing and pacing device.
11. The system of any of embodiments 7-10, wherein the left ventricular pacing devices are powered by transmission of ultrasonic signals independently of an on-board battery.
12. The system of any of embodiments 7-11, wherein the left ventricular pacing devices further include a sensor to sense one or more of spontaneous left ventricular electrical activity, blood temperature, blood velocity, and blood pressure within the heart or an actuator to provide cardiac stimulation or pacing.
13. The system of any of embodiments 7-12, wherein the plurality of intra-cardiac left ventricular pacing devices are implantable in one or more main branches of a coronary sinus on the left ventricle.
14. The system of any of embodiments 1-13, wherein the second implantable device comprises a subcutaneously implantable central unit comprising a processing unit, the processing unit including one or more processors and memory, an ultrasonic transducer to transmit and receive ultrasonic signals to the first intra-cardiac implantable device.
15. The system of any of embodiments 1-14, further comprising at least an additional intra-cardiac implantable device implantable in an atrium or a ventricle of a heart, comprising an ultrasonic transducer operative to receive ultrasonic signals, and pacing circuitry operative to convert an acoustic signal into an electrical signal to stimulate or control a cardiac rhythm; and

wherein the second implantable device comprises a subcutaneously implantable central unit comprising a processing unit, the processing unit including one or more processors and memory, an ultrasonic transducer to transmit and receive ultrasonic signals to the first and the additional intra-cardiac implantable devices.

16. The system of embodiment 15, wherein the first intra-cardiac implantable device comprises a right atrial sensing and pacing device implantable in a right atrium of the heart, and the additional intra-cardiac implantable device comprises a right ventricular sensing and pacing device implantable in a right ventricle of the heart.
17. The system of embodiment 16, wherein the right atrial sensing and pacing device and the right ventricular sensing and pacing device are operable to communicate with each other via ultrasonic signals independently of the central unit.
18. The system of any of embodiments 1-17, wherein the first intra-cardiac implantable device comprises a ventricular pacing device implantable in a left ventricle of the heart, and further comprising at least an additional intra-cardiac implantable device comprising a plurality of further left ventricular pacing devices implantable in a left ventricle of the heart; and

wherein the second implantable device comprises a subcutaneously implantable central unit comprising a processing unit, the processing unit including one or more processors and memory, an ultrasonic transducer to transmit and receive ultrasonic signals to the first and the plurality of left ventricular intra-cardiac implantable devices.

19. The system of embodiment 18, further comprising a right ventricular sensing and pacing device implantable in a right ventricle and operable to transmit instructions and energy to the left ventricular pacing devices via ultrasonic signals independently of the central unit.
20. The system of any of embodiments 18-19, wherein the ultrasonic transducers of the left ventricular pacing devices are arranged in an array, and the central unit is operable to transmit signals to the left ventricular implantable devices with a controlled phase delay.
21. The system of embodiment 20, wherein array of the ultrasonic transducers has an area less than 1.5×1.5 mm².
22. The system of any of embodiments 20-21, wherein each ultrasonic transducer is spaced about 250 μm from an adjacent ultrasonic transducer.
23. The system of any of embodiments 20-22, wherein the array of ultrasonic transducers has a center frequency of less than about 5 MHz.
24. The system of any of embodiments 20-23, wherein each of the ultrasonic transducers is operative with a pressure efficiency of about 1 kPa/V
25. The system of any of embodiments 20-24, wherein the array of the ultrasonic transducers is operable with a half power beam width of about 20° and a pressure at a focal point about 36 times larger than a pressure at an individual ultrasonic transducer at a same distance.
26. The system of any of embodiments 1-25, wherein the first intra-cardiac implantable device further includes an array of ultrasonic transducers.
27. The system of any of embodiments 1-26, wherein the first intra-cardiac implantable device further includes a sensor operative to monitor cardiac contractility and kinesis in a right atrium or a right ventricle of a heart.
28. The system of any of embodiments 1-27, wherein the first intra-cardiac implantable device is operative to detect a beat-to-beat spatial distribution of a heart.
29. The system of any of embodiments 1-28, wherein the second implantable device comprises a central unit operative to determine an origin of ventricular tachycardia or fibrillation transmitted from the first intra-cardiac implantable device and a plurality of additional intra-cardiac implantable devices implantable in a heart.
30. The system of any of embodiments 1-29, wherein the second implantable device comprises a central unit operative to determine an occurrence of a cardiac arrhythmia in the heart from the first intra-cardiac implantable device implanted in a right ventricle of the heart.
31. The system of any of embodiments 1-30, wherein the second implantable device comprises a central unit operative to transmit an instruction to provide ventricular pacing to the first intra-cardiac implantable device.
32. The system of any of embodiments 1-31, wherein the second implantable device comprises a central unit operative to determine an occurrence of bradycardia from the first intra-cardiac implantable device implanted in a right atrium of the heart.
33. The system of any of embodiments 1-32, wherein the second implantable device comprises a central unit operative to transmit an instruction to provide atrial-synchronized ventricular pacing to the first intra-cardiac implantable device implanted in a right ventricle of the heart.
34. The system of any of embodiments 1-33, wherein the second implantable device comprises a central unit operative to transmit an instruction to the first intra-cardiac implantable device implanted in a left ventricle to provide left ventricular pacing for cardiac resynchronization therapy.
35. The system of any of embodiments 1-34, wherein the second implantable device comprises a central unit operative to provide instructions to the first intra-cardiac implantable device to provide one or more of anti-tachycardia pacing, anti-bradycardia pacing, arrhythmia correction, resynchronization, and defibrillation of a heart.
36. The system of any of embodiments 1-35, wherein the second implantable device comprises a central unit implantable in a pocket between chest muscles.
37. The system of any of embodiments 1-36, the second implantable device comprises a central unit, and further comprising a subcutaneously implantable sensing lead, the central unit in communication with the sensing lead to detect a heart rate and a cardiac arrhythmia of a heart.
38. The system of any of embodiments 1-37, wherein the second implantable device comprises a central unit, and further comprising a subcutaneously implantable defibrillation lead, the central unit in communication with the defibrillation lead to provide a defibrillation shock to a heart.
39. The system of any of embodiments 1-38, wherein the first intra-cardiac implantable device has a volume less than about 1 cm³.
40. The system of any of embodiments 1-39, wherein the first intra-cardiac implantable device includes a fixation system configured to affix the device to myocardium of the heart.
41. The system of any of embodiments 1-40, wherein the first intra-cardiac implantable device is embeddable in a stent implantable in a branch of a coronary sinus.
42. The system of any of embodiments 1-41, wherein each of the ultrasonic transducers comprises a piezoelectric microelectromechanical transducer.
43. The system of embodiment 42, wherein the ultrasonic transducer comprises a piezoelectric membrane.
44. The system of any of embodiments 42-43, wherein the piezoelectric membrane is aluminum nitride.
45. The system of any of embodiments 1-44, wherein each of the ultrasonic transducers comprises a piezoelectric membrane suspended between opposed electrodes.
46. The system of any of embodiments 1-45, wherein each of the ultrasonic transducers comprises a piezoelectric membrane suspended to deflect out of a plane of the piezoelectric membrane.
47. The system of any of embodiments 1-46, wherein each of the ultrasonic transducers has a resonant frequency of about 3 MHz with a bandwidth of about 1 MHz.
48. The system of any of embodiments 1-47, wherein each of the ultrasonic transducers is operative to generate a surface pressure of about 12 kPa/V.
49. The system of any of embodiments 1-48, wherein the pacing circuitry comprises circuitry operative to detect an acoustic pressure signal and convert the detected acoustic pressure signal into an electrical signal.
50. The system of any of embodiments 1-49, wherein the pacing circuitry comprises:

a piezoelectric ultrasonic transducer operative at a resonant frequency to convert an incoming acoustic pressure wave at the resonant frequency into a voltage signal;

a load capacitor chargeable by the voltage signal; and

a pacing electrode electrically connected to the load capacitor to generate an electrical stimulus to the heart.

51. The system of embodiment 50, wherein the pacing circuitry further comprises a switch or a relay electrically connected to an acoustic receiver operative to receive an acoustic signal at a further frequency, the switch or relay electrically connected between the load capacitor and the pacing electrode to connect the pacing electrode to the load capacitor upon receipt of the acoustic signal at the further frequency.
52. The system of any of embodiments 1-51, wherein each of the implantable devices includes a processing unit including one or more processors and memory.
53. The system of any of embodiments 1-52, wherein each of the implantable devices includes a core unit comprising a microcontroller unit, a field programmable gate array (FPGA), or both a microcontroller unit and an FPGA operative to execute communication, processing, and networking tasks.
54. The system of embodiment 53, wherein the core unit includes one or both of a serial peripheral interface (SPI) and an inter integrated circuit (I2C) interface to control communications between the microcontroller, the FPGA, the ultrasonic transducer, and the pacing circuitry.
55. The system of any of embodiments 1-54, wherein each of the implantable devices includes a core unit comprising one or more logic devices to control the ultrasonic transducer and the pacing device, the one or more logic devices including small-scale integrated circuits, programmable logic arrays, programmable logic devices, masked-programmed gate arrays, field programmable gate arrays, and application specific integrated circuits.
56. The system of any of embodiments 1-55, wherein the first intra-cardiac implantable device further includes one or more sensors or actuators, the sensors or actuators comprising one or more of a heart rate sensor, blood temperature sensor, blood velocity sensor, and blood pressure sensor, cardiac stimulator, or cardiac pacer.
57. The system of any of embodiments 1-56, wherein the first intra-cardiac implantable device is rechargeable via an ultrasonic signal transmitted from the central unit or an external acoustic source.
58. The system of any of embodiments 1-57, wherein the first intra-cardiac implantable device includes a battery and is operable to harvest power for recharging the battery from one or more of transmitted ultrasonic signals and an acoustic noise source.
59. The system of embodiment 58, wherein the acoustic noise source includes heart beats or a human voice.
60. A system for monitoring and controlling heart rhythms, comprising:

a network of implantable devices comprising at least:
a right atrial intra-cardiac sensing and pacing device implantable in a right atrium of a heart comprising an ultrasonic transducer operative to transmit ultrasonic signals; and
a right ventricular intra-cardiac sensing and pacing device implantable in a right ventricle of the heart, comprising an ultrasonic transducer operative to receive ultrasonic signals from the right atrial sensing and pacing device to stimulate or control the cardiac rhythm, and pacing circuitry operative to convert an ultrasonic signal into an electrical signal to stimulate or control a cardiac rhythm
   61. The system of embodiment 60, wherein the right atrial sensing and pacing device and the right ventricular sensing and pacing device are operable to communicate with each other via ultrasonic signals.
   62. The system of any of embodiments 60-61, wherein the right atrial sensing and pacing device is operative to sense spontaneous atrial electrical activity in the heart if an intrinsic heart rate is above a predetermined pacing lower rate, or pace the right atrium if the intrinsic heart rate is below the predetermined pacing lower rate.
   63. The system of any of embodiments 60-62, wherein the right atrial sensing and pacing device is further operative to transmit an ultrasonic signal to the right ventricular sensing and pacing device to trigger a determined atrio-ventricular delay interval, and the right ventricular sensing and pacing device is operative to inhibit pacing if the spontaneous ventricular electrical activity of the heart occurs within the delay interval and to deliver pacing if the spontaneous ventricular activity does not occur within the delay interval.
   64. The system of embodiment 63, wherein the delay interval ranges from about 100 ms to about 400 ms.
   65. A system for monitoring and controlling heart rhythms, comprising:

a network of implantable devices comprising at least:
a plurality of intra-cardiac left ventricular pacing devices implantable in a left ventricle of the heart, each left ventricular pacing device comprising an ultrasonic transducer operative to receive ultrasonic signals, and pacing circuitry operative to convert an ultrasonic signal into an electrical signal to stimulate or control a cardiac rhythm; and
a right ventricular sensing and pacing device implantable in a right ventricle of the heart comprising an ultrasonic transducer operative to transmit ultrasonic signals to the plurality of intra-cardiac left ventricular pacing devices to stimulate or control the cardiac rhythm.
   66. The system of embodiment 65, wherein the right ventricular sensing and pacing device is operative to transmit instructions via ultrasonic signals to pace each of the plurality of left ventricular pacing devices.
   67. The system of any of embodiments 65-66, wherein the right ventricular sensing and pacing device is operative to transmit instructions to focus pacing the left ventricular pacing device closest to a determined origin of arrhythmia of the heart.
   68. The system of any of embodiments 65-67, wherein each of the left ventricular pacing devices is powered by ultrasonic signals transmitted from the right ventricular sensing and pacing device.
   69. The system of any of embodiments 65-68, wherein the left ventricular pacing devices are powered by transmission of ultrasonic signals independently of an on-board battery.
   70. The system of any of embodiments 65-69, wherein the left ventricular pacing devices are implantable in one or more main branches of a coronary sinus on the left ventricle of the heart.
   71. The system of any of embodiments 65-70, wherein the left ventricular pacing devices further include a sensor to sense one or more of spontaneous left ventricular electrical activity, blood temperature, blood velocity, and blood pressure within the heart.
   72. A system for monitoring and controlling heart rhythms, comprising:

a network of implantable devices comprising at least:
a plurality of intra-cardiac implantable devices each implantable in an atrium or a ventricle of a heart and comprising an ultrasonic transducer operative to receive ultrasonic signals, and pacing circuitry operative to convert an ultrasonic signal into an electrical signal to stimulate or control a cardiac rhythm; and
a subcutaneously implantable central unit comprising a processing unit, the processing unit including one or more processors and memory, an ultrasonic transducer to transmit and receive ultrasonic signals to the plurality of intra-cardiac implantable devices.
   73. The system of embodiment 72, wherein a first device of the intra-cardiac implantable devices comprises a right atrial sensing and pacing device implantable in a right atrium of the heart, and a second device of the intra-cardiac implantable devices comprises a right ventricular sensing and pacing device implantable in a right ventricle of the heart.
   74. The system of embodiment 73, wherein the right atrial sensing and pacing device and the right ventricular sensing and pacing device are operable to communicate with each other via ultrasonic signals independently of the central unit.
   75. The system of embodiment 72-74, wherein each of the plurality of intra-cardiac implantable devices comprises a ventricular pacing device implantable in a left ventricle of the heart,
   76. The system of embodiment 75, further comprising a right ventricular sensing and pacing device implantable in a right ventricle operable to transmit instructions and energy to the left ventricular pacing devices via ultrasonic signals.
   77. The system of any of embodiments 75-76, wherein the right ventricular sensing and pacing device is operable to transmit instructions and energy to the left ventricular pacing devices via ultrasonic signals independently of the central unit.
   78. A method of monitoring and controlling heart rhythms comprising:

implanting the network of implantable devices of any of embodiments 1-77 in a subject in need thereof; and

sensing or controlling a heart rhythm by at least the first intra-cardiac implantable device or one of the intra-cardiac sensing and pacing devices.

79. The method of embodiment 78, further comprising monitoring cardiac contractility and kinesis of the heat by sensing acceleration and beat-to-beat spatial distribution obtained from each of the implantable devices.
80. The method of any of embodiments 78-79, wherein the network includes one or more additional intra-cardiac implantable devices including an ultrasonic transducer, and further comprising determining an origin of ventricular tachycardia or fibrillation within the heart from ultrasonic signals transmitted by each of the intra-cardiac implantable devices.
81. The method of embodiment 80, further comprising transmitting an instruction for defibrillation of the heart from the second implantable device.
82. The method of embodiment 81, wherein the system includes an implanted defibrillation lead and the instruction for defibrillation is transmitted from the second implantable device to the defibrillation lead.
83. The method of any of embodiments 78-82, further comprising transmitting an instruction to the first intra-cardiac implantable device to control pacing of the heart.
84. The method of any of embodiments 78-83, wherein the network includes one or more additional intra-cardiac implantable devices including an ultrasonic transducer, and further comprising transmitting an instruction from the second implantable device to one or more of the intra-cardiac implantable devices to provide anti-tachycardia pacing or anti-bradycardia pacing of the heart.
85. The method of any of embodiments 78-84, wherein the network includes one or more additional intra-cardiac implantable devices including an ultrasonic transducer, and further comprising transmitting an instruction to one or more of the intra-cardia implantable devices to provide resynchronization of the heart.
86. A device for monitoring and controlling heart rhythms, comprising:

an intra-cardiac implantable device implantable in an atrium or a ventricle of a heart comprising an ultrasonic transducer operative to receive ultrasonic signals, and pacing circuitry operative to convert an acoustic signal into an electrical signal to stimulate or control a cardiac rhythm.

87. The device of embodiment 86, wherein the pacing circuitry comprises circuitry operative to detect an acoustic pressure wave and convert the detected acoustic pressure wave into an electrical signal.
88. The device of any of embodiments 86-87, wherein the pacing circuitry comprises:

a piezoelectric ultrasonic transducer operative at a resonant frequency to convert an incoming acoustic pressure wave at the resonant frequency into a voltage signal;

a load capacitor chargeable by the voltage signal; and

a pacing electrode electrically connected to the load capacitor to generate an electrical stimulus to the heart.

89. The device of embodiment 88, wherein the pacing circuitry further comprises a switch or a relay electrically connected to an acoustic receiver operative to receive an acoustic signal at a further frequency, the switch or relay electrically connected between the load capacitor and the pacing electrode to connect the pacing electrode to the load capacitor upon receipt of the acoustic signal at the further frequency.
90. The device of any of embodiments 86-89, wherein the ultrasonic transducer comprise a piezoelectric microelectromechanical transducer.
91. The device of any of embodiments 86-90, wherein the ultrasonic transducer comprises a piezoelectric membrane of aluminum nitride.
92. The device of any of embodiments 86-91, wherein the ultrasonic transducer comprises a piezoelectric membrane suspended to deflect out of a plane of the piezoelectric membrane.
93. A micro-electromechanical piezoelectric ultrasonic transducer device comprising:

a piezoelectric layer having first and second opposed surfaces, the piezoelectric layer supported along a fixed boundary by a substrate, the piezoelectric layer deflectable out of a plane;

a first electrode disposed on the first surface of the piezoelectric layer, and a second electrode disposed on the second surface of the piezoelectric layer;

wherein the piezoelectric layer is operative to deflect out of the plane by an incoming pressure wave and to deflect out of the plane by a voltage applied across the first and second electrodes; and

circuitry in electrical communication with the first and second electrodes to convert a deflection of the piezoelectric layer into an electric signal or to apply a voltage across the first and second electrodes to force a deflection of the piezoelectric layer.

94. The device of embodiment 93, wherein:

the piezoelectric layer is operative at a resonant frequency to convert an incoming pressure wave at the resonant frequency into a voltage signal; and

the circuitry comprises a load capacitor chargeable by the voltage signal, and a pacing electrode electrically connected to the load capacitor to generate an electrical stimulus to the heart.

95. The device of any of embodiments 93-94 wherein the circuitry further comprises a switch or a relay electrically connected to an acoustic receiver operative to receive an acoustic signal at a further frequency, the switch or relay electrically connected between the load capacitor and the pacing electrode to connect the pacing electrode to the load capacitor upon receipt of the acoustic signal at the further frequency.
96. The device of any of embodiments 93-95, wherein each of the piezoelectric layer has a resonant frequency of about 3 MHz with a bandwidth of about 1 MHz.
97. The device of any of embodiments 93-96, wherein the piezoelectric layer s is operative to generate a surface pressure of about 12 kPa/V.

### DESCRIPTION OF THE DRAWINGS

Reference is made to the following detailed description taken in conjunction with the accompanying drawings in which:

FIG. 1 is a schematic illustration of an embodiment of a system for controlling and monitoring a heart;

FIG. 2 is a comparison of attenuation of ultrasonic and radio frequency (RF) waves in human muscle;

FIG. 3A is a schematic cross sectional view of an embodiment of a micro-electro-mechanical system aluminum nitride piezoelectric micromachined ultrasonic transducer (MEMS AlN PMUT);

FIG. 3B is a finite element method (FEM) simulation model of the PMUT of FIG. 3A illustrating a membrane mode of vibration;

FIG. 3C is a FEM simulation model of the PMUT illustrating a sound pressure field;

FIG. 4A is a graph illustrating PMUT membrane displacement vs. frequency based on both an analytical model and a FEM model;

FIG. 4B is a graph of surface pressure vs. frequency generated by an incoming acoustic wave;

FIG. 5 is a schematic block diagram of an embodiment of the architecture of a sensing and pacing device;

FIG. 6 is an exploded schematic illustration of an embodiment of a sensing and pacing device;

FIG. 7 is a graph of predicted performance of an ultrasonic wideband transducer communication protocol;

FIG. 8 is a schematic block diagram of an embodiment of software architecture of a sensing and pacing device;

FIG. 9 is a circuit diagram of an embodiment of a zero-power architecture capable of producing a pacing electrical stimulus upon detection of an incoming acoustic signature;

FIG. 10A is a graph of sensitivity vs. frequency for an FEM model of a PMUT operated in fluid; and

FIG. 10B is a graph of maximum power extractable vs. distance from a PMUT for a 720 mW/cm² power density transmitted through soft tissues using an ˜26 kHz ultrasonic link.

### DETAILED DESCRIPTION

A system and method to monitor and control heart rhythms using ultrasonic signals, including providing pacing and defibrillation therapy, are provided. Embodiments include a wireless multi-site network of implantable sensing and pacing devices (SPDs) and/or pacing devices (PDs) in which data can be exchanged between the devices through digitally modulated ultrasonic pulses that are generated and detected through miniaturized piezoelectric ultrasonic transducers.

In some embodiments, the system includes at least a first intra-cardiac implantable device implantable in an atrium or a ventricle of a heart and a second implantable device implantable in the heart or subcutaneously. Each device includes an ultrasonic transducer operative to receive and/or transmit ultrasonic signals. One or more devices include pacing circuitry operative to convert an acoustic signal into an electrical signal to stimulate or control a cardiac rhythm. The ultrasonic transducers can be based on micromachined piezoelectric aluminum nitride (AlN) technology.

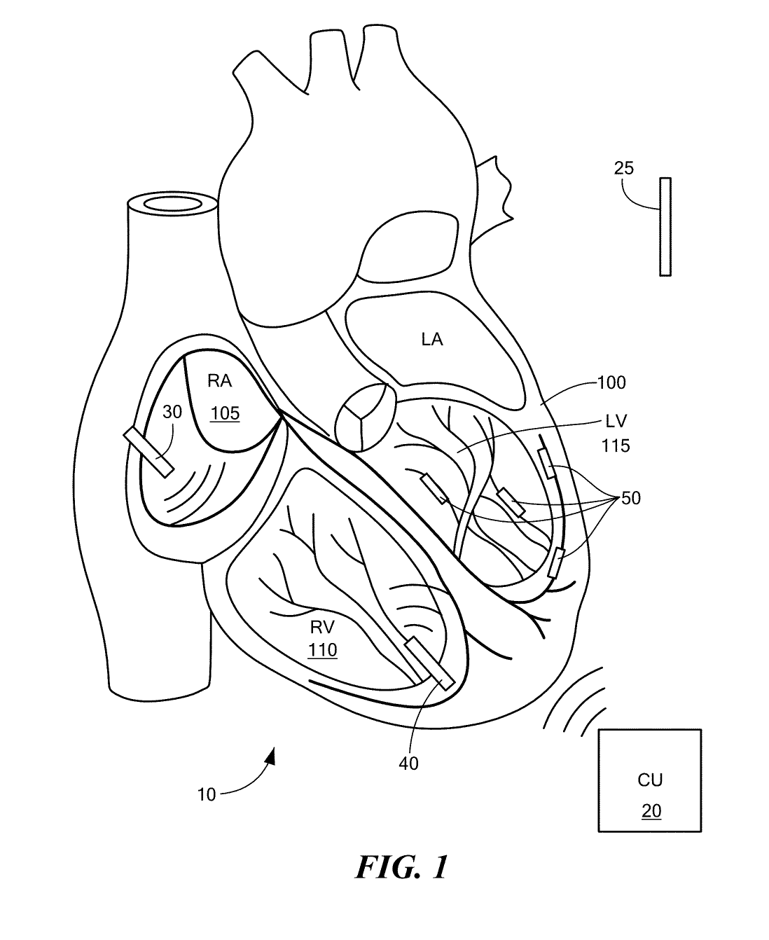

Referring to the embodiment of FIG. 1, the system 10 can include a subcutaneously implantable central unit (CU) 20, which can control and monitor other devices of the network. The other devices can include two intra-cardiac sensing and pacing devices (SPDs) 30, 40 implantable in the right atrium 105 and right ventricle 110 of a heart 100, respectively. A number of additional intra-cardiac pacing devices (PDs) 50 can be implantable in the left ventricle 115 of the heart, for example, in the main branches of the coronary sinus on the epicardial surface of the left ventricle. The system can also include a subcutaneously implantable sensing and/or defibrillation lead 25. The wireless sensing and pacing devices, the pacing devices, and the central unit can form a wireless network in which data can be exchanged between the different devices through digitally modulated ultrasonic pulses that are generated and detected through miniaturized piezoelectric transducers. The ultrasonic transducers can be based on micro-electromechanical system (MEMS) piezoelectric, aluminum nitride (AlN) technology. Use of wireless ultrasonic transmissions can overcome limitations of classical wireless communications based on electromagnetic radio frequency (RF) propagation, which are power-hungry, unreliable, and possibly not safe in human tissues.

In some embodiments, the sensing and pacing device (SPD) can include sensing, pacing, processing, and ultrasonic communication capabilities. The device can be based on mm-sized reprogrammable electronics and be integrated with the ultrasonic micromachined transducers. In some embodiments, the pacing device (PD) can be a passive, mm³-sized, battery-less device including circuitry capable of pacing the heart by converting an ultrasonic wave transmitted by an SPD in the right ventricle into a conventional electric pacing signal. In some embodiments, the sensing and pacing devices and/or the pacing devices can include a battery for providing power. In some embodiments, energy harvesters within the devices can be capable of recharging the batteries in less than 6 hours through focused ultrasound beams.

The system can provide a number of capabilities. For example, in some embodiments, the system can provide multi-site sensing and pacing. The system can interconnect, based on wireless control data links, a subcutaneously implantable central unit, which can be a defibrillator control unit, with multiple leadless sensing and pacing devices and/or pacing devices. In this manner, the system can be based on multiple, wirelessly networked, intra-cardiac sensors/actuators distributed over multiple sensing and pacing sites. In contrast, prior art implantable cardioverter defibrillators have a wired pacing/sensing lead implanted in the right ventricle.

In some embodiments, the system can provide multi-site wireless pacing. In some embodiments, the system can employ multiple, wirelessly coordinated and controlled sensing and actuation sites. For example, one sensing and pacing device can be implanted in the right atrium, and another sensing and pacing device can be implanted in the right ventricle. As a further example, multiple passive, wirelessly-controlled and -powered pacing devices can be placed at multiple sites in the left ventricle. The passive pacing devices can be in communication with a sensing and pacing device, which can be implanted, for example, in the right ventricle. In contrast, existing prior art leadless pacemakers are able to perform pacing without the need for wires based on only one individual sensing site.

In some embodiments, the system can provide synchronized adaptive pacing. For example, multiple pacing devices can include actuators to pace the heart, in a synchronized fashion, at various locations. Pacing timing can be controlled in real time based on information gathered by multiple cardiac sensors that interact wirelessly, through ultrasounds, in a distributed fashion, with the pacing devices.

The system can employ ultrasonic wireless connectivity. Ultrasonic, digitally modulated, impulsive waveforms can carry information and control messages and create a wireless ultrasonic network among the different devices of the system. In some embodiments, wireless connectivity between an implantable central control unit and implantable intra-cardiac devices can be provided through wireless links based on ultrasonic carrier waves. Ultrasonic waves are safer, more energy efficient, more secure, and reliable than radio-frequency (RF) waves in cardiac tissues. Compared to RF electromagnetic waves used in commercial wireless technologies like Bluetooth, WiFi, or MICS, ultrasonic waves are absorbed significantly less by human tissues (i.e., 8-16 dB for a 10-20 cm link at 1 MHz, vs 60-90 dB at 2.45 GHz as used in Bluetooth). Therefore, tissue heating is much reduced, which results in significantly longer duration of the batteries when used, and prevents absorption of microwaves by biological tissues.

The devices of the system can employ micromachined ultrasonic transducers for use in the wireless ultrasonic communications. In some embodiments, ultrasonic waves can be generated and detected by ultra-wideband, low-power transducers based on micro-electro-mechanical system (MEMS) piezoelectric, aluminum nitride technology. Such transducers can have a reduced size and weight and improved energy efficiency when compared to prior art bulk piezoelectric transducers.

In some embodiments, ultrasonic transducers within the devices can utilize the electromechanical properties of aluminum nitride (AlN) ultra-thin piezoelectric films in micro-electro-mechanical (MEMS) ultrasonic transducers. Such transducers can have high sensitivity, adjustable wide bandwidth (>1 MHz), low transmit voltage (suitable for low power electronics) and intrinsic acoustic impedance match to cardiac tissues in a miniaturized form factor. The same MEMS structure can work both as a transmitter and a receiver of data and energy. The resulting miniaturized piezoelectric transducers can enable ultra low power and reliable ultrasonic wireless communication in tissues and ultrasonic recharge of batteries.

In some embodiments, the system can employ ultrasonic wireless recharging and energy harvesting. For example, ultrasonic transducers in one or more of the devices can be used to harvest power to recharge batteries from environmental acoustic noise (e.g., from noise created by heart beats, a human voice, and other acoustic and mechanical sources of noise). Ultrasonic transducers in the devices can be used to wirelessly recharge the devices through focused ultrasonic beams, which can be externally generated. Since exposure of human tissues to ultrasounds is safer than RF, the FDA allows significantly higher intensity for ultrasonic waves (720 mW/cm²) in tissues as compared to RF (10 mW/cm² limit), i.e., almost two orders of magnitude. This makes it possible to recharge batteries much faster through ultrasound than using RF. In some embodiments, MEMS ultrasonic energy harvesters can allow a device to fully charge (assuming 20% efficiency) a deeply implanted 3.6V 200 mAh battery (such as those used in prior art pacemakers) in less than 6 hours through a focused external generator of ultrasounds.

Based on information collected by the network of intra-cardiac sensors, and on their pacing capabilities, a variety of capabilities can be enabled by embodiments of the system. For example, the system can provide real-time and multi-site monitoring of left and right ventricular function. The system can provide detection of the origin of life-threatening arrhythmias to provide effective anti-tachycardia therapy. The devices can include sensors to detect blood temperature, velocity, and pressure for heart failure monitoring. Multi-site pacing of the left ventricle to ensure real-time adaptive cardiac resynchronization therapy can be provided. The system can sense spontaneous left ventricular electrical activity (as occurring in case of life-threatening cardiac arrhythmia) and ensure that high-rate anti-tachycardia pacing is delivered through the pacing device that is spatially closest to the focus of arrhythmia origin. The system can determine cardiac rhythm acceleration time and mutual location within the heart, thus providing real-time insights into cardiac contractility.

As noted above, wireless networking and recharging of the implantable devices described herein is based on the propagation of ultrasounds rather than RF waves. Acoustic waves in the ultrasonic spectral regime can be used to carry digital data (for control or telemetry) among multiple implantable devices. These waveforms can be generated and detected through miniaturized micro-electro-mechanical systems (MEMS) piezoelectric ultrasonic transducers (PMUT) in the subcutaneous and intra-cardiac implantable devices. The ultrasonic wireless technology is safer, more secure, and consumes less energy than traditional RF-based standards. The system can result in smaller battery size and/or longer time between procedures to change batteries. In some embodiment, the pacing devices can be batteryless, described further below.

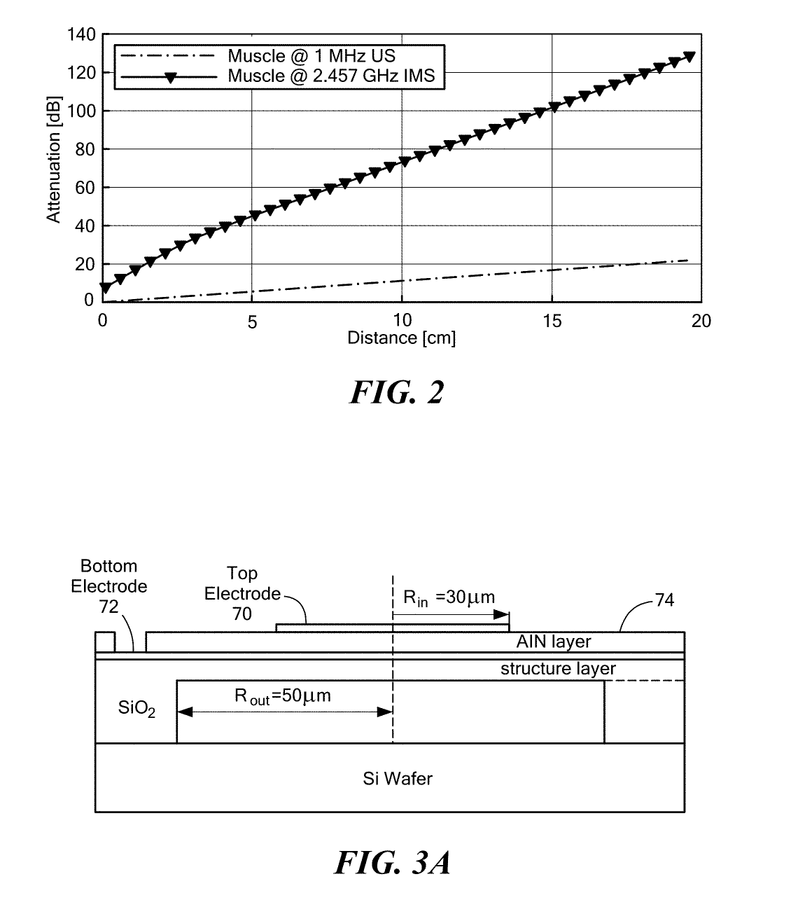

Compared to radio-frequency (RF) electromagnetic waves (microwaves) used in Bluetooth or WiFi, ultrasonic waves have advantages for use in cardiac implantable devices. Ultrasonic waves have significantly lower absorption by biological tissues, e.g., 8-16 dB for a 10-20 cm link at 1 MHz, vs. 60-90 dB at 2.45 GHz as used in Bluetooth. FIG. 2. Therefore, tissue heating is much reduced, which makes propagation safer. Ultrasounds are the safest mode of transmission of energy, as long as acoustic power dissipation in tissues is limited to predefined safety levels. Moreover, transmission power can be orders-of-magnitude lower, and therefore implantable battery-powered devices can last longer and/or be smaller in size. Related to this, the FDA also allows much higher intensity for ultrasonic waves (720 mW/cm²) in tissues as compared to RF (10 mW/cm²), i.e., almost two orders of magnitude higher. When one factors in the lower absorption/attenuation, wireless recharging of batteries through ultrasonic waves can in some embodiments be orders of magnitude faster than with RF.

Additionally, multi-path propagation is easier to resolve because of the lower propagation speed of acoustic waves. Therefore small transducers that operate at low frequencies can be used. Such small transducers are also easier to couple to human tissues than RF antennas, which instead need to operate at high frequencies. Also, ultrasonic propagation is largely confined in the body; therefore, ultrasonic intra-body networks are inherently more secure with respect to eavesdropping and jamming attacks. Further, there are no or fewer electromagnetic compatibility concerns with a crowded RF spectrum. The Ultrasonic wideband (UsWB) technology eliminates conflicts with existing RF communication systems and overcrowded RF environments.

In some embodiments, ultrasonic power transmission schemes can be used to safely enable wireless battery charging functionalities. On-board ultrasonic transducers can also be used to enable acoustic localization and tracking functionalities, which can have better accuracy than their RF-based counterpart because of the low propagation speed of sound in human tissues. In some embodiments, the UsWB transmission scheme can implement a carrierless impulse-based integrated physical layer and medium access control scheme that can flexibly trade off performance for power consumption. In some embodiments, the UsWB transmission scheme can be shown to achieve, for bit error rates lower than 10⁻⁶ over 20 cm links in tissue, either (i) high-data rate transmissions up to 700 kbit/s at a transmit power of −14 dBm (40 μW), or (ii) low-data rate and lower-power transmissions down to −21 dBm (8 μW) at 70 kbit/s.

In some embodiments, ultrasonic transmissions can be provided by microelectromechanical systems (MEMS) micromachined ultrasound transducers (MUTs). MEMS based ultrasound transducers can offer advantages such as increased bandwidth, flexible geometries, natural acoustic match with aqueous media, reduced voltage requirements, and potential for integration with supporting electronic circuits.

In some embodiments, micro-machined ultrasound transducers based on thin film piezoelectric membranes (PMUTs) can be used. PMUTs are advantageous, as they do not require a small gap and a DC bias voltage to achieve efficient transduction. In some embodiments, aluminum nitride (AlN) piezoelectric films can be used. A high quality ultra-thin AlN film can be directly deposited on silicon substrates by a low-temperature sputtering process, enabling the fabrication of ultra-low volume MEMS resonant structures with good electromechanical performance. PMUTs based on thin-film AlN can provide good performance in terms of efficiency, sensitivity and high density integration. Furthermore, the microfabrication process used for AlN MEMS devices is compatible with subsequent CMOS processes to enable their monolithic integration with low power CMOS electronics, which is suitable for the implementation of ultra-miniaturized, high performance, high density, and low power sensing and wireless communication platforms suitable for implantable cardiac devices and for use with high-performance, CMOS-compatible physical, chemical and biological sensors. AlN-based PMUTs can show higher receiving sensitivity than more conventional lead zirconate titanate (PZT)-based devices, because of the smaller dielectric constant of the AlN piezoelectric material.

An array of micro-machined ultrasonic aluminum nitride MEMS transducers can be provided that meets suitable CU-to-SPD and/or -PD and SPD-to-PD communication requirements in terms of transducer size, center frequency, bandwidth, and efficiency, while simultaneously providing focusing and beamforming capabilities. In some embodiments, an array of transducers can be arranged in an area less than about 1.5×1.5 mm². In some embodiments, an array of transducers can be arranged with a center frequency less than about 5 MHz. In some embodiments, an array of transducers can be arranged with a bandwidth of about 1 MHz. In some embodiments, an array of transducers can be arranged with an efficiency of about kPa/V.

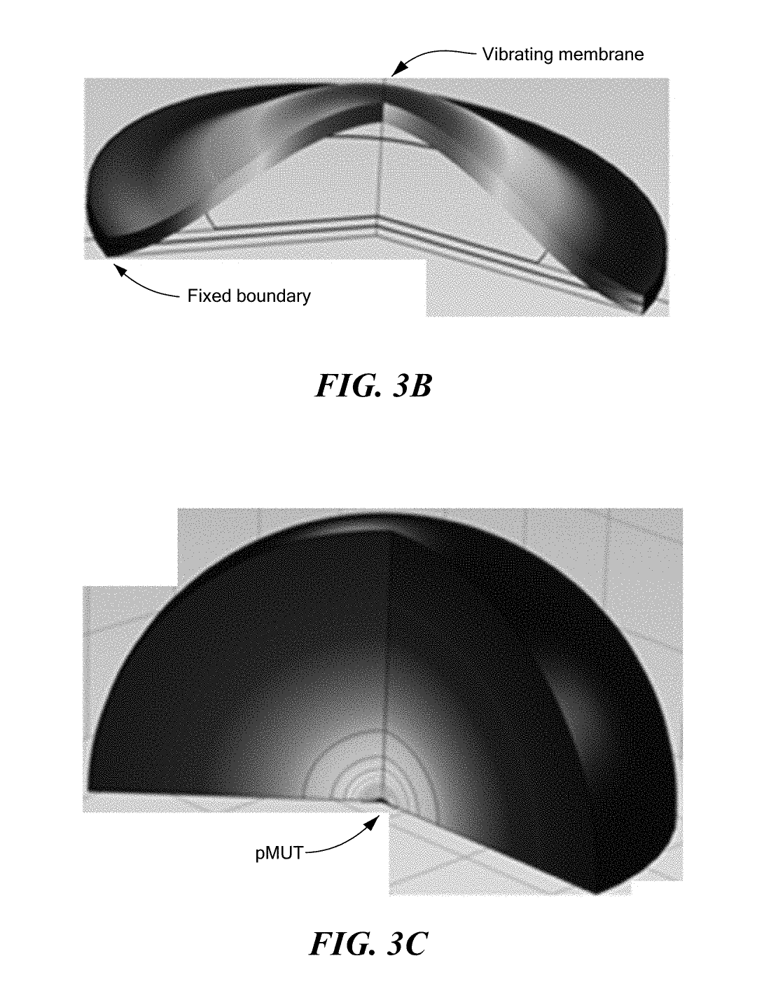

An embodiment of an individual AlN PMUT suitable for use in a phased array is shown in FIGS. 3A, 3B, and 3C. The same MEMS structure can work both as a transmitter and a receiver. As a transmitter, the electric field between a top electrode 70 and a bottom electrode 72 induces a longitudinal stress in a suspended AlN piezoelectric layer 74, due to the inverse piezoelectric effect, which forces the membrane to deflect out of plane launching a pressure wave into the adjacent medium. As a receiver, charge between the electrodes is generated due to direct piezoelectric effect when longitudinal stress (membrane deflection) is induced by an incident wave.

It will be appreciated that other piezoelectric materials can be used in some embodiments if desired, depending on the application. For example, lead zirconate titanate (PZT) has been investigated for PMUTs due to its high piezoelectric coefficient, hence transduction efficiency. However, a high temperature fabrication process is needed (around 800° C.) for the production of PZT films, which makes this material incompatible with CMOS processes. Moreover, environmental and health hazards associated with lead raise concerns regarding the use of PZT in implantable medical devices.

In some embodiments, the piezoelectric material can be aluminum nitride, gallium nitride, aluminum scandium nitride, aluminum magnesium nitride, gallium arsenide, lead zirconium titanium oxide, lead zirconium titanium, molybdenum sulfide, aluminum zirconium magnesium nitride, aluminum erbium magnesium nitride, quartz, silicon oxide, ammonium, potassium hydrogen phosphate, rochelle salt, lithium niobate, silicon selenite, germanium selenite, lithium sulfate, antimony sulfoiodide, barium titanate, calcium barium titanate, lead titanate zirconate, apatite, bimorphs, gallium phosphate, lanthanum gallium silicate, lead scandium tantalate, lithium tantalate, polyvinylidene fluoride, potassium sodium tartrate, lead lanthanum zirconate titanate, lead magnesium niobate, lithium nibonate, lead titanate, or zinc oxide.

Further description of devices employing piezoelectric materials can be found in WO 2017/066195, WO 2015/161257, WO 2015/012914, and WO 2014/138376, the disclosures of which are incorporated by reference herein.

Other ultrasonic transducers can be used in some embodiments if desired. For example, capacitive MUTs (CMUTs) can provide satisfactory performance as both ultrasound transmitters and receivers. In CMUTS, however, electrostatic transduction requires use of small gaps and high DC bias voltages (typically exceeding 100V), which makes the use of CMUTs in implantable devices less optimal when compared to PMUTs.

In some embodiments, two intra-cardiac implantable sensing and pacing devices 30, 40 (SPDs) can be provided, for implantation in the right atrium (RA-SPD) and in the right ventricle (RV-SPD), respectively. The devices can provide data processing, sensing, leadless pacing and wireless communication capabilities.

In some embodiments, the SPD can provide a flexible platform for sensing, processing, networking, and pacing. Many or all functionalities, including communications, networking, sensing/pacing, and processing functionalities, can be reconfigurable and software-defined. The SPD can have a small and compact form factor compatible with the state of the art in-chip integration to provide these functionalities. The SPD can be made of ultra-low-power, highly integrated, and reprogrammable components. The SPD can have ultrasonic wireless recharging and energy harvesting capabilities. The SPD can embed miniaturized MEMS ultrasonic transducers as transceivers and energy harvesters.

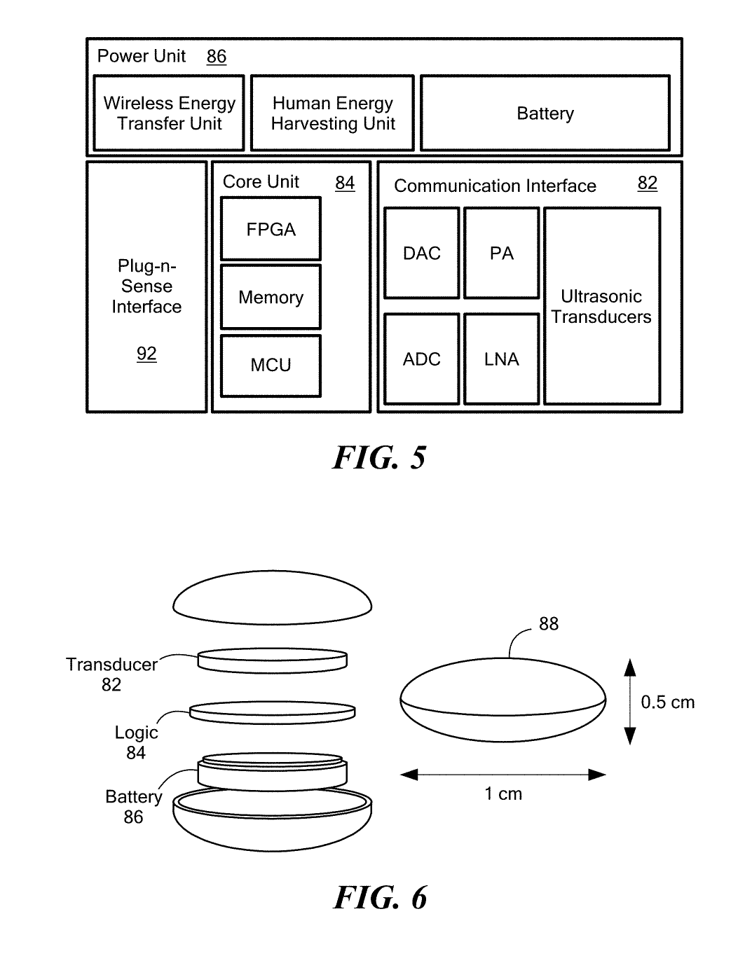

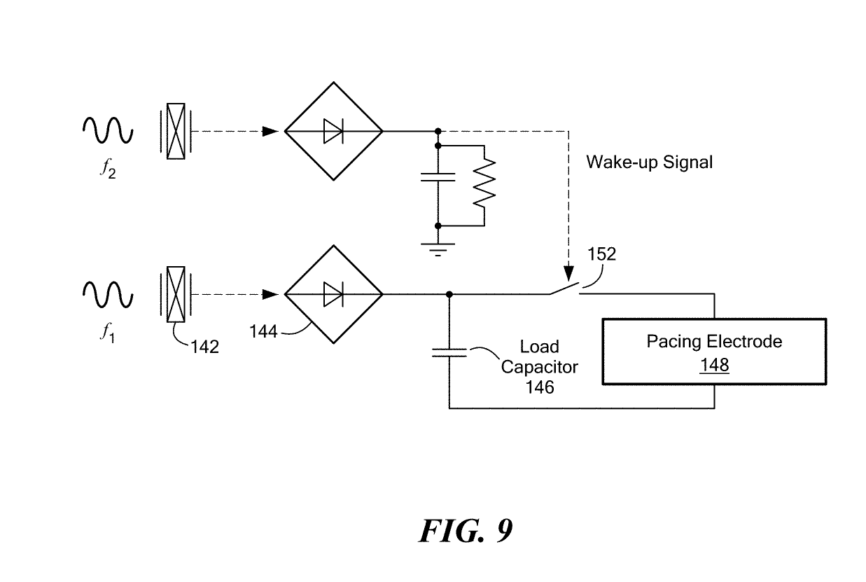

Referring to FIGS. 5 and 6, in some embodiments, each sensing and pacing device can include a core unit that includes mm-size ultra low-power processing units, such as a microcontroller and one or more logic devices to control the ultrasonic transducer and the pacing device). A reconfigurable programmable digital circuit and low power microcontroller can offer hardware and software reprogrammability to support cardiac processing algorithms. In some embodiments, the one or more logic devices can include small-scale integrated circuits, programmable logic arrays, programmable logic devices, masked-programmed gate arrays, field programmable gate arrays, and application specific integrated circuits. In some embodiments, the devices can have zero static power consumption when idle, and can be woken up on demand (described further below with respect to FIG. 9). Referring to FIG. 6, the components, an ultrasonic transducer or communication unit 82, the logic device(s) or core unit 84, and a battery or power unit 86, can be provided in a suitable case or housing 88, which can be made of a biocompatible material. The device can be miniaturized, having a volume on the order of 1 cm³.

FIG. 5 shows an embodiment of a block functional architecture of SPD hardware. In this embodiment, the hardware can include a core unit 84, a communication interface 82, a power unit 86, and a sensing and pacing interface 92. In the embodiment illustrated, the core-unit of the SPD can include mm-size low-power processing units, an MCU and an FPGA, as well as a non-volatile memory. The miniaturized FPGA can host the physical (PHY) layer and some time-critical media access control (MAC) functionalities of the wireless protocol stack. The core unit can also enable flexible hardware implementation of cardiac-related algorithms, such as an arrhythmia detection algorithms, without sacrificing energy efficiency.

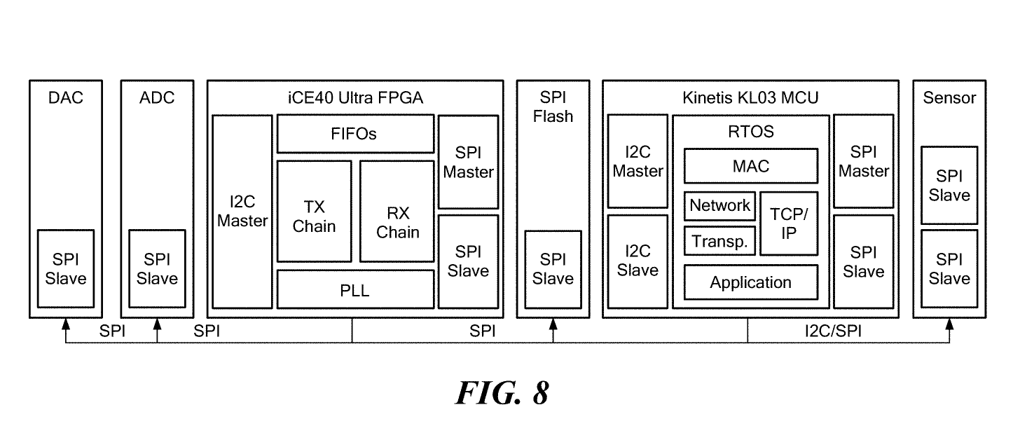

Referring also to FIG. 8, in some embodiments, the FPGA can include a set of integrated hardened IP cores, including two SPI and two I2C blocks that can operate both as master and slaves to enable connectivity with virtually any sensors, data converters, memories and MCUs. A set of digital signal processing (DSP) functional blocks can be provided to off-load computationally intensive arrhythmia detection operations to the FPGA.

In some embodiments, the SPD's MCU can control data processing and execution of software-defined functionalities to implement flexible and reconfigurable upper-layer protocols. In some embodiments, the MCU can include memory, such as flash memory and/or SRAM. The MCU can employ a real time operating system (RTOS), which can run in a resource constrained environment, to support software and programming bare-metal applications. A variety of embedded RTOSs are commercially available, such as μTasker, which is suitable for single chip applications as described herein. The MCU can connect directly to the FPGA, to sensors, and to data converters, ADC and DAC, through an SPI module, a low-power UART module and a high-speed I2C module. Analog inputs can be connected to the ADC. The MCU can be provided in a millimeter-size packaging and have low-power consumption.

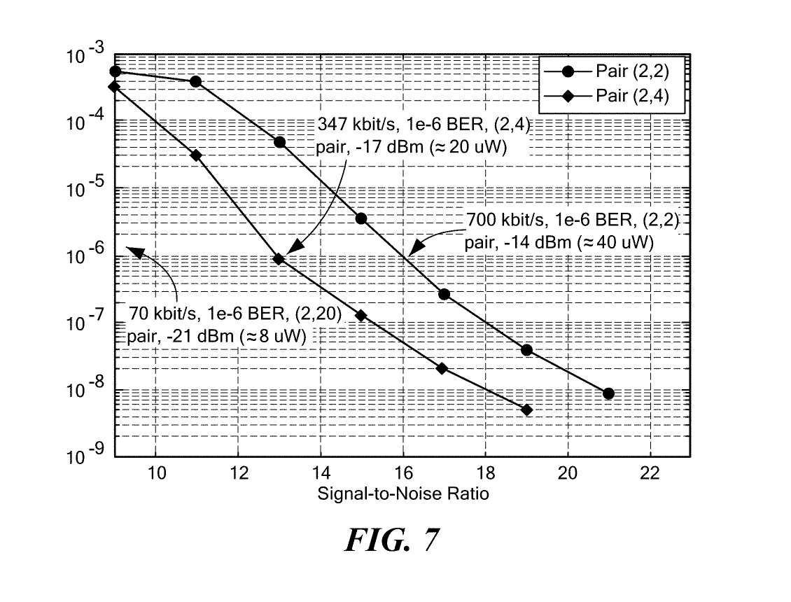

The communication interface 82 can enable ultrasonic wireless connectivity through data converters, power and low-noise amplifiers, and custom ultrasonic transducers. For example, the communication interface can include a receiver (Rx) and a transmitter (Tx) chain. The Rx chain can include a low-noise amplifier (LNA) and an analog-to-digital converter (ADC) to amplify and digital-convert received signals. The Tx chain can embed a digital-to-analog converter (DAC) and a power amplifier (PA) to analog-convert and amplify the digital waveforms before transmission. The Tx and Rx chains can control transmitting and receiving acoustically software-generated digital streams through the ultrasonic transducers. In some embodiments, the SPD can communicate over a bandwidth of about 1 MHz centered at 1 MHz range. The 1 MHz bandwidth enables transmission of pulses of duration 200 ns, which enable reliable low-power communications in the presence of strong multipath, multi-user interference, and ease synchronization and localization. In some embodiments, an ultrasonic wideband (UsWB) protocol can be used. UsWB is an impulse-based ultrasonic transmission and multiple access technique based on transmitting short information-bearing carrierless ultrasonic pulses, following a pseudo-random adaptive time-hopping pattern with a superimposed spreading code of adaptive length. Impulsive transmission and spread-spectrum encoding combat the effects of multipath and scattering and introduce waveform diversity among interfering transmissions. Information is carried through pulse position modulation (PPM). A predicted performance of UsWB protocol implemented in the FPGA is shown in FIG. 7.

The FPGA top-level module can instantiate Tx and Rx chain blocks implementing the ultrasonic wideband communication functionalities, a set of first-in-first-out (FIFO) memory queue blocks, a pair of SPI Master/Slave blocks, an I2C Master block, and a PLL block. The logic can be driven by an external system clock signal inputted to one of the FPGA's pins.

The SPD can embed an array of ultrasonic transducers, such as micro-machined ultrasonic aluminum nitride MEMS transducers as described above, to meet the integration requirements and provide focusing and beamforming capabilities. The wireless communication interface can implement suitable communication and networking schemes. Known communication and networking schemes can be provided that are fully software-defined and composable through a set of modular libraries.

The interface 92 of the SPD can enable the inclusion of additional components, for example, to accommodate actuators or electrodes for sensing and electrical stimulation. The interface can be a flexible interface capable of receiving plug-in components. Sensors, such as blood temperature, pressure, and velocity sensors, can be provided. In some embodiments, conventional actuators or electrodes for stimulation, pacing, sensing, such as blood temperature, pressure, and velocity, and the like can be used.

As noted above, the SPD can enable implementation of cardiac-related algorithms. For example, in some embodiments, the SPDs can provide defibrillation. In some embodiments, the RV-SPD can be provided with an active fixation system based on tines that can embed into the myocardium. In addition to the cosmetic advantage, the leadless design and lack of a surgically created pocket eliminate or minimize the complications associated with conventional pacemaker implantation. The SPD can be implantable in both the right atrium (RA-SPD) and the right ventricle (RV-SPD). The SPD can have real-time wireless telemetry and control capabilities based on ultrasonic data links (UsWB). The SPD can be controlled directly, communicate in real-time, and be recharged wirelessly as needed by the CU. The SPD can trigger pacing in the left ventricle (LV) by sending energy and timing control signals to one or more the pacing devices in the LV.

The RA-SPD and RV-SPD can serve as sensing and pacing electrodes for real time, dual-chamber anti-bradycardia pacing. The RA-SPD can sense spontaneous atrial electrical activity (if intrinsic heart rate is above the programmed pacing lower rate) or pace the right atrium (if intrinsic heart rate is below the programmed pacing lower rate). This sensing/pacing activity can be sent through the ultrasonic wireless link to the ventricular SPD, triggering a programmable atrio-ventricular delay. For example, the delay can range from about 100 ms to about 400 ms. If spontaneous ventricular electrical activity occurs within this time interval, the RV-SPD can detect the intrinsic cardiac signal and inhibit pacing. On the contrary, if spontaneous ventricular activity does not occur within the pre-specified delay, the ventricular SPD can deliver pacing and send this information to the RA-SPD. In this embodiment, the dual-chamber anti-bradycardia pacing can be independent of the central unit. Due to close proximity of the RA-SPD and the RV-SPD, and the absence of air on the path (i.e., lungs), the devices can reliably communicate at minimal energy consumption and radiated power.

At least one and preferably a plurality of left-ventricular pacing devices (LV-PDs) 50 can be provided. In some embodiments, at least 3 to 5 pacing devices are provided. In some embodiments the left ventricular devices can be passive or batteryless pacing devices (PPDs) and can be powered by the right ventricular sensing and pacing device 40 through ultrasonic waves. In this manner, the PPDs can pace the heart when powered. In some embodiments, the pacing devices can have a size on the order of a few mm³. In some embodiments, the pacing devices can be embedded in stents that can be co-axially mounted onto an inflatable balloon of a standard balloon angioplasty catheter and implanted in the main branches of the coronary sinus, on the epicardial surface of the left ventricle. In some embodiments, the implantation procedure can be similar to that commonly used for routine coronary angioplasty.

In some embodiments, at least 3 to 5 LV-PDs can be implanted into the branches of the coronary sinus and be therefore able to provide multi-site pacing of the left ventricle. Pacing via LV-PDs can occur upon ultrasonic energy transfer by the RV-SPD and be determined and controlled by pacing algorithms that reside within the RV-SPD processing unit. Therefore, in some embodiments, this pacing can be independent of the subcutaneous control unit (CU), which can have the advantage of allowing low-power ultrasonic communication and energy transfer through a distance of a few inches (typically less than 5 inches) and across fluids (blood) and tissues (cardiac muscle). In contrast, using an external unit for energy transfer to or communication with the RV-PPD can require ultrasounds to pass through organs with significant air content (lungs) and travel over much longer distances (typically greater than 10 inches).

In some embodiments, the LV-PDs can be powered, for example, by micro supercapacitors and provided with data storage, sensing, and a micro-processor unit. In some embodiments, such powered LV-PDs can be capable of sensing spontaneous left ventricular electrical activity (as in case of life-threatening cardiac arrhythmia) and can ensure that high-rate anti-tachycardia pacing can be delivered through the PD spatially closest to the focus of arrhythmia origin. In some embodiments, powered LV-PDs can determine their acceleration time and mutual location within the heart, thus providing real-time insights into cardiac contractility. In some embodiments, powered LV-PDs can include on-board sensors to provide information on blood temperature, velocity or pressure used to predict heart failure.

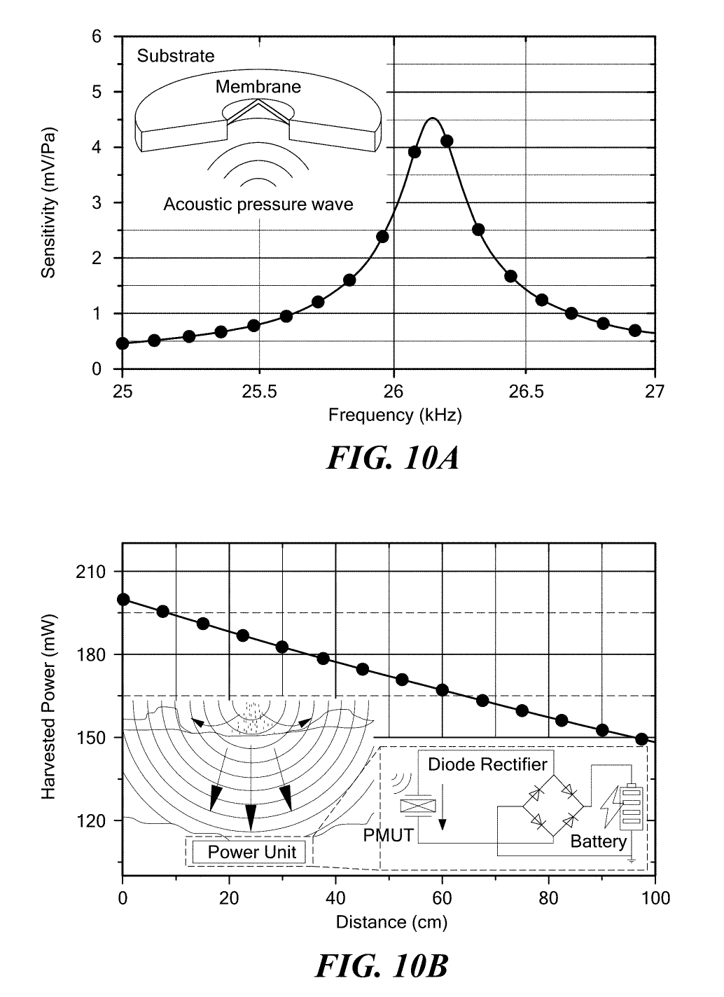

In some embodiments, the architecture of the PPDs can include a zero-power acoustic receiver capable of detecting a specific “pacing” acoustic pressure signal signature emitted by the SPD, harvesting its energy and converting it into a voltage pulse of, for example, 1˜5 V needed to perform pacing. In some embodiments, the receiver can detect a “pacing” acoustic pressure signal signature of interest and discriminate it in the presence of a noisy background by MEMS enabled filtering. Referring to FIG. 9, an embodiment of a zero power acoustic receiver can be triggered by an acoustic pressure signature consisting of two tones (for example, 100 s Pa amplitude) at two specific frequencies (for example, f₁=26 kHz and f₂=30 kHz) emitted by two PMUTs included in the SPD. The first stage of the receiver can be a high sensitivity AlN PMUT 142, with a resonance frequency f₁, that efficiently converts the acoustic pressure wave at frequency f₁ into a voltage signal (for example, 1˜5 V amplitude) at the same frequency. The resonant nature of the AlN PMUT (see FIG. 10) can enable filtering of the “pacing” signal frequency from the entire spectrum. The generated AC voltage signal is then rectified, for example, using a typical diode rectifier 144, and used to charge a load capacitor 146. One terminal of the capacitor is directly connected to the first terminal of the pacing electrode 148 while the other terminal of the capacitor is connected to the second terminal of the pacing electrode through a MEMS relay 152 or other switching device. The state of the MEMS relay is controlled by the rectified voltage at the output of an analog acoustic receiver tuned to the second tone (at frequency f₂) contained in the acoustic pressure signal signature. When the MEMS relay is in open state (as shown) the load capacitor is physically disconnected (through an air gap) from the pacing electrode, enabling the achievement of extremely low leakage current through the pacing site when the pacing pressure signature is absent. When the MEMS relay is triggered to the closed state (i.e., when the acoustic pressure tone at frequency f₂ is received), the voltage stored in the load capacitor 146 is applied to the pacing electrode 148 generating the electrical stimulus.

In some embodiments, the central unit (CU) can be a subcutaneously implantable device with ultrasonic networking capabilities that can control can control and coordinate the other sensing and pacing devices in the network and can control delivery of a defibrillation shock to a heart. In some embodiments, the central device can control wireless recharging through ultrasound transmissions of the sensing and pacing devices implanted in the right atrium and the ventricles.

In some embodiments, the central unit (CU) can employ a programmable system-on-chip (SOC) architecture. The SOC can include programmable logic such as a FPGA integrated with a processor, which can be substantially similar to that described above with respect to the SPDs. The programmable logic can implement lower-level processing functionalities (including UsWB), and the processor can implement higher-level algorithms and communication protocols. The CU can include an ultrasonic communication unit, which includes a power amplifier and low-noise amplifier interfaced with ultrasonic transducers in the transmit and receive chains, respectively. A power unit including a battery can be connected with the on-board circuitry of the SOC and communication unit. The CU can include a suitable housing or case, which can be made of a biocompatible material, for example, titanium.

The CU can be implanted in a suitable location in a patient's body, such as within the chest. For example, in some embodiments, the CU can be implanted posterolaterally in a surgically created pocket created by blunt dissection between the anterior surface of the serratus anterior and the posterior surface of the latissmus dorsi, over the left sixth rib, between the mid and the anterior axillary lines.

In some embodiments, the CU device can sense intrinsic heart rate and detect cardiac arrhythmia through a single sensing lead 25 (FIG. 1) that can be implanted subcutaneously, for example, on the left parasternal line, outside the chest. The sensing lead can also be provided with electrodes that can, together with a device canister, determine multiple vectors for surface ECG sensing. The lead can also be provided with a subcutaneous electrode for use with the CU to provide a high-energy defibrillation shock. For example, in some embodiments, the subcutaneous electrode can have a proximal and distal ring electrode on each side of a defibrillation coil electrode (for example, a 3 inch (8 cm) defibrillation coil electrode). Other sensing and/or defibrillation electrode configurations can be provided. The lead 25 can be any conventional sensing and/or defibrillation lead.

Algorithms implemented in the SOC can determine a pacing and/or defibrillation therapy to be provided by the system. For example, pacing can be provided at a rate of 50 beats per minute up to 30 seconds after a shock for defibrillation. Noise filtering and pre-programmed algorithms for arrhythmia detection and discrimination can be provided to ensure that a life-threatening arrhythmia can be treated, and shocks for benign arrhythmia mimicking fatal arrhythmia (inappropriate therapy) are minimized. After confirmation of a life-threatening arrhythmia, the system can deliver anti-tachycardia pacing through wireless pacing via the RV-SPD and/or a higher energy defibrillation shock between the coil on the parasternal lead and device canister.

Compared to radio-frequency (RF) electromagnetic waves used in Bluetooth or WiFi, ultrasonic waves are significantly less absorbed by human tissues; therefore, tissue heating is much reduced, which makes propagation safer for humans. For this reason, the FDA allows much higher intensity for ultrasonic waves (720 mW/cm²) in tissues as compared to RF (10 mW/cm²). This feature can be used to wirelessly recharge a battery of a device through ultrasonic waves. In some embodiments, a high output power acoustic transducers (charger) can be used to generate a pressure signal and a high sensitivity AlN PMUT can be used to detect the pressure wave and harvest its energy to charge the SPD battery. In some embodiments, wireless charging can employ a charger transmitting a maximum FDA approved intensity (720 mW/cm²) at a distance of up to about 1 meter from the receiver. In some embodiments, a charging transmitter can be located at a greater distance or a lesser distance. In some embodiments, a battery, such as a 3.6 V 200 mAh implantable battery, can be fully charged in less than 6 hours. In some embodiments, the AlN MEMS PMUT can be used for the implementation of integrated energy harvesters capable of scavenging energy from acoustic noise, such as a human voice in a range of about 100 Hz to about 5 kHz, or heart beats. For example an AlN PMUT with a radius of ˜500 μm, operated in fluid, could be used to harvest acoustic noise in a narrow bandwidth (˜1 kHz) centered at ˜3 kHz.

Further description of devices employing ultrasonic transducers can be found in WO 2016/123069, WO 2016/123047, and WO 2016/112166, the disclosures of which are incorporated by reference herein.

The intra-cardiac sensing and pacing devices in the right ventricle and atrium and intra-cardiac pacing devices in the left ventricle can communicate and coordinate sensing and pacing actions with one another and with the subcutaneous central unit in real time by means of an ultrasonic intra-body network. This can allow the system to achieve a variety of capabilities, including:
Monitor cardiac contractility and kinesis by integrating data from sensor acceleration and beat-to-beat spatial distribution of the sensor network into the heart (i.e., from the sensing and pacing devices in the right atrium and right ventricle, and from the pacing devices in the left ventricle.
Detect the origin of ventricular tachycardia or fibrillation via beat-to-beat multi-site analysis and wirelessly transmit data to the subcutaneous central unit for prompt defibrillation therapy.
Provide leadless anti-tachycardia pacing for rapid-rate life-threatening ventricular arrhythmia via the network of pacing devices (one sensing and pacing device in the right ventricle, one sensing and pacing device in the right atrium, and multiple pacing devices in the left ventricle) that can react based on pre-defined programmable options stored in the central unit.
Perform leadless anti-bradycardia pacing through a system of multiple electrodes on the sensing and pacing devices and the pacing devices that can coordinate with each other to provide synchronized atrio-ventricular pacing.
Deliver leadless multisite cardiac resynchronization therapy based on the network of sensing and pacing devices and pacing devices with distributed control that creates a multi-point map of the electromechanical activation pattern of the heart and adaptively react to provide optimized resynchronization.
Provide defibrillation therapy for life-threatening cardiac arrhythmia upon automatic arrhythmia detection. In contrast to standard implantable defibrillators, the system can provide automatic arrhythmia detection via the right ventricular sensing and pacing device. This device can wirelessly send heart rhythm specifications to the subcutaneous central unit. In some embodiments, the central unit can merge key rhythm information from the right ventricular sensing and pacing device and electrocardiographic traces from the subcutaneous defibrillation lead when present to provide optimized arrhythmia detection. After confirming a life-threatening condition, the system can provide high-rate ventricular pacing through the left ventricular pacing devices (LV-PDs), for anti-tachycardia pacing (ATP). In case of ATP failure, the system can deliver one or more high-energy defibrillation shocks.
Allow synchronized atrio-ventricular pacing to treat symptomatic bradycardia. The right atrial sensing and pacing device (RA-SPD) can sense spontaneous atrial rhythm (or provide low-energy pacing in case of lack of spontaneous rhythm) and can wirelessly send this sensing and pacing event information to the right ventricular sensing and pacing device (RV-SPD). The RV-SPD can then activate a pre-programmed sensing window and wait for spontaneous ventricular electrical activity. In case of lack of spontaneous ventricular rhythm, the RV-SPD can provide atrial-synchronized ventricular pacing.
Provide left ventricular (LV) pacing for cardiac resynchronization therapy (CRT) in heart failure patients. In patients requiring continuous bi-ventricular pacing, the RV-SPD can wirelessly transfer ultrasonic energy and activate the LV-PPDs for pacing. LV pacing onset can be synchronous, anticipated or delayed with respect to RV pacing according to the specific cardiac physiology of the individual patient.

### EXAMPLES

### Finite Element Method Simulations

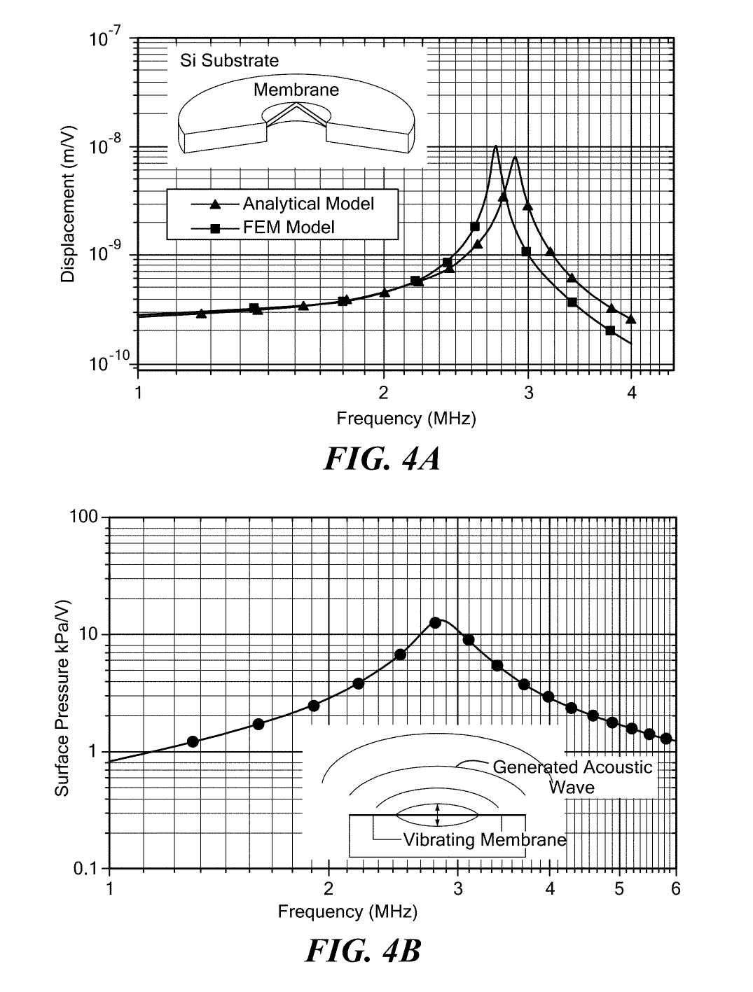

A finite element method (FEM) simulation of the AlN MEMS ultrasonic transducer shown in FIGS. 3A-3C was conducted to validate the analytical model. The simulations indicate that the AlN PMUT has a resonance frequency of ˜3 MHz with a bandwidth of ˜1 MHz, when operated in fluid, and generates a surface pressure of ˜12 kPa/V. See FIGS. 4A and 4B.

FEM simulation of a ˜26 kHz AlN PMUT operated in fluid (FIG. 10A) indicates that a maximum sensitivity of ˜4.5 mV/Pa can be achieved in the narrow-band of interest. State of the art MEMS relays are characterized by threshold voltage values as low as 100s mV. Therefore, in this embodiment, the load capacitor can be charged to ˜1 V (voltage level suitable for pacing) upon detection of a ˜200 Pa acoustic pressure signal. Similarly, the reception of a relatively low amplitude (˜10 s Pa) acoustic pressure tone at frequency f₂ is sufficient to activate the MEMS switch and trigger the pacing voltage pulse.

### Wireless Charging Example

As an example of wireless charging using a vibrating AlN piezoelectric membrane (PMUT) in a central unit (CU) or sensing and pacing device (SPD), if a typical diode rectifier with ˜50% efficiency is employed, and assuming matched load impedance, for a ˜26 kHz power transfer link, (receiver PMUT radius r˜200 μm, with peak sensitivity of ˜4.5 mV/Pa (FIG. 10A)) employing a charger transmitting at maximum FDA approved intensity (720 mW/cm²) and placed at a distance ˜1 m from the receiver (assuming soft tissue as medium with ˜0.5 dB/(cm×MHz)), it will be possible to extract a maximum power, Pₗᵢₘ, of ˜150 mW (FIG. 10B). This would provide a maximum time to full charge (assuming 20% efficiency loss) of less than 6 hours for a 3.6 V 200 mAh implantable battery.

As used herein, “consisting essentially of” allows the inclusion of materials or steps that do not materially affect the basic and novel characteristics of the claim. Any recitation herein of the term “comprising,” particularly in a description of components of a composition or in a description of elements of a device, can be exchanged with “consisting essentially of” or “consisting of”

It will be appreciated that the various features of the embodiments described herein can be combined in a variety of ways. For example, a feature described in conjunction with one embodiment may be included in another embodiment even if not explicitly described in conjunction with that embodiment.

To the extent that the appended claims have been drafted without multiple dependencies, this has been done only to accommodate formal requirements in jurisdictions which do not allow such multiple dependencies. It should be noted that all possible combinations of features which would be implied by rendering the claims multiply dependent are explicitly envisaged and should be considered part of the invention.

The present invention has been described in conjunction with certain preferred embodiments. It is to be understood that the invention is not limited to the exact details of construction, operation, exact materials or embodiments shown and described, and that various modifications, substitutions of equivalents, alterations to the compositions, and other changes to the embodiments disclosed herein will be apparent to one of skill in the art.

What is claimed is:

1. A system for monitoring and controlling heart rhythms, comprising:
a network of implantable devices comprising at least:
a first intra-cardiac implantable device implantable in an atrium or a ventricle of a heart comprising an ultrasonic transducer operative to receive ultrasonic signals, and pacing circuitry operative to convert an acoustic signal into an electrical signal to stimulate or control a cardiac rhythm; and
a second implantable device comprising an ultrasonic transducer operative to transmit ultrasonic signals to the first intra-cardiac implantable device to stimulate or control the cardiac rhythm.

2. The system of claim 1, wherein the first intra-cardiac implantable device comprises a right ventricular intra-cardiac implantable sensing and pacing device implantable in a right ventricle of the heart; and
the second implantable device comprises a right atrial sensing and pacing device implantable in a right atrium of the heart.

3. The system of claim 2, wherein the right atrial sensing and pacing device is operative to sense spontaneous atrial electrical activity in the heart if an intrinsic heart rate is above a predetermined pacing lower rate, or pace the right atrium if the intrinsic heart rate is below the predetermined pacing lower rate.

4. The system of claim 1, wherein the first intra-cardiac implantable device comprises a plurality of intra-cardiac left ventricular pacing devices implantable in a left ventricle of the heart; and
the second implantable device comprises a right ventricular sensing and pacing device implantable in a right ventricle of the heart.

5. The system of claim 4, wherein the right ventricular sensing and pacing device is operative to transmit instructions via ultrasonic signals to pace each of the plurality of left ventricular pacing devices.

6. The system of claim 5, wherein the right ventricular sensing and pacing device is operative to transmit instructions to focus pacing the left ventricular pacing device closest to a determined origin of arrhythmia of the heart.

7. The system of claim 4, wherein each of the left ventricular pacing devices is powered by ultrasonic signals transmitted from the right ventricular sensing and pacing device.

8. The system of claim 4, wherein the left ventricular pacing devices further include a sensor to sense one or more of spontaneous left ventricular electrical activity, blood temperature, blood velocity, and blood pressure within the heart or an actuator to provide cardiac stimulation or pacing.

9. The system of claim 1, further comprising at least an additional intra-cardiac implantable device implantable in an atrium or a ventricle of a heart, comprising an ultrasonic transducer operative to receive ultrasonic signals, and pacing circuitry operative to convert an acoustic signal into an electrical signal to stimulate or control a cardiac rhythm; and
wherein the second implantable device comprises a subcutaneously implantable central unit comprising a processing unit, the processing unit including one or more processors and memory, an ultrasonic transducer to transmit and receive ultrasonic signals to the first and the additional intra-cardiac implantable devices.

10. The system of claim 9, wherein the first intra-cardiac implantable device comprises a right atrial sensing and pacing device implantable in a right atrium of the heart, and the additional intra-cardiac implantable device comprises a right ventricular sensing and pacing device implantable in a right ventricle of the heart.

11. The system of claim 1, wherein the first intra-cardiac implantable device comprises a ventricular pacing device implantable in a left ventricle of the heart, and further comprising at least an additional intra-cardiac implantable device comprising a plurality of further left ventricular pacing devices implantable in a left ventricle of the heart; and
wherein the second implantable device comprises a subcutaneously implantable central unit comprising a processing unit, the processing unit including one or more processors and memory, an ultrasonic transducer to transmit and receive ultrasonic signals to the first and the plurality of left ventricular intra-cardiac implantable devices.

12. The system of claim 1, wherein the second implantable device comprises a central unit operative to determine an origin of ventricular tachycardia or fibrillation transmitted from the first intra-cardiac implantable device and a plurality of additional intra-cardiac implantable devices implantable in a heart.

13. The system of claim 1, wherein the second implantable device comprises a central unit operative to determine an occurrence of a cardiac arrhythmia in the heart from the first intra-cardiac implantable device implanted in a right ventricle of the heart.

14. The system of claim 1, wherein the second implantable device comprises a central unit operative to transmit an instruction to provide ventricular pacing to the first intra-cardiac implantable device.

15. The system of claim 1, wherein the second implantable device comprises a central unit operative to provide instructions to the first intra-cardiac implantable device to provide one or more of anti-tachycardia pacing, anti-bradycardia pacing, arrhythmia correction, resynchronization, and defibrillation of a heart.

16. The system of claim 1, the second implantable device comprises a central unit, and further comprising a subcutaneously implantable sensing lead or defibrillation lead, the central unit in communication with the sensing lead to detect a heart rate and a cardiac arrhythmia of a heart or to provide a defibrillation shock to a heart.

17. The system of claim 1, wherein each of the ultrasonic transducers comprises a piezoelectric microelectromechanical transducer comprising a piezoelectric membrane suspended between opposed electrodes and to deflect out of a plane of the piezoelectric membrane.

18. The system of claim 17, wherein the piezoelectric membrane is aluminum nitride.

19. The system of claim 1, wherein the pacing circuitry comprises circuitry operative to detect an acoustic pressure signal and convert the detected acoustic pressure signal into an electrical signal, comprising
a piezoelectric ultrasonic transducer operative at a resonant frequency to convert an incoming acoustic pressure wave at the resonant frequency into a voltage signal;
a load capacitor chargeable by the voltage signal; and
a pacing electrode electrically connected to the load capacitor to generate an electrical stimulus to the heart.

20. The system of claim 1, wherein:
the first intra-cardiac implantable device is rechargeable via an ultrasonic signal transmitted from the central unit or an external acoustic source, or
the first intra-cardiac implantable device includes a battery and is operable to harvest power for recharging the battery from one or more of transmitted ultrasonic signals and an acoustic noise source.

21. A system for monitoring and controlling heart rhythms, comprising:
a network of implantable devices comprising at least:
a right atrial sensing and pacing device implantable in a right atrium of a heart comprising an ultrasonic transducer operative to transmit ultrasonic signals; and
a right ventricular intra-cardiac sensing and pacing device implantable in a right ventricle of the heart, comprising an ultrasonic transducer operative to receive ultrasonic signals from the right atrial sensing and pacing device to stimulate or control the cardiac rhythm, and pacing circuitry operative to convert an ultrasonic signal into an electrical signal to stimulate or control a cardiac rhythm

22. A system for monitoring and controlling heart rhythms, comprising:
a network of implantable devices comprising at least:
a plurality of intra-cardiac left ventricular pacing devices implantable in a left ventricle of the heart, each left ventricular pacing device comprising an ultrasonic transducer operative to receive ultrasonic signals, and pacing circuitry operative to convert an ultrasonic signal into an electrical signal to stimulate or control a cardiac rhythm; and
a right ventricular sensing and pacing device implantable in a right ventricle of the heart comprising an ultrasonic transducer operative to transmit ultrasonic signals to the plurality of intra-cardiac left ventricular pacing devices to stimulate or control the cardiac rhythm.

23. A system for monitoring and controlling heart rhythms, comprising:
a network of implantable devices comprising at least:
a plurality of intra-cardiac implantable devices each implantable in an atrium or a ventricle of a heart and comprising an ultrasonic transducer operative to receive ultrasonic signals, and pacing circuitry operative to convert an ultrasonic signal into an electrical signal to stimulate or control a cardiac rhythm; and
a subcutaneously implantable central unit comprising a processing unit, the processing unit including one or more processors and memory, an ultrasonic transducer to transmit and receive ultrasonic signals to the plurality of intra-cardiac implantable devices.

24. A method of monitoring and controlling heart rhythms comprising:
implanting the network of implantable devices of claim 1 in a subject in need thereof; and
sensing or controlling a heart rhythm by at least the first intra-cardiac implantable device.
